(19) United States
(12) Patent Application Publication
(10) Pub. No.: US 2022/0039912 A1
(43) Pub. Date: Feb. 10, 2022

(54) ENDOSCOPE CLEANING DEVICE

(71) Applicant: Covidien LP, Mansfield, MA (US)

(72) Inventors: Justin J. Thomas, New Haven, CT (US); Jacob C. Baril, Norwalk, CT (US); Matthew A. Dinino, Newington, CT (US); Nicolette R. LaPierre, Windsor Locks, CT (US); Roy J. Pilletere, Middletown, CT (US); Saumya Banerjee, Hamden, CT (US); Garrett P. Ebersole, Hamden, CT (US)

(21) Appl. No.: 17/353,932

(22) Filed: Jun. 22, 2021

Related U.S. Application Data
(60) Provisional application No. 63/063,640, filed on Aug. 10, 2020.

(51) Int. Cl. A61B 90/70; A61B 1/00; A61B 1/018

(52) U.S. Cl. CPC A61B 90/70; A61B 1/0014; A61B 2090/701; A61B 1/00096; A61B 1/018; A61B 1/0011

(57) ABSTRACT

A cleaning device enables cleaning of a lens of an endoscope during a surgical procedure to maintain a clear image without having to remove the endoscope from the patient's body. The cleaning device is disposed on an elongate shaft of a surgical instrument such that a lens of an endoscope may be wiped against the cleaning device to remove debris from the lens.

### CROSS-REFERENCE TO RELATED APPLICATIONS

This application claims the benefit of and priority to U.S. Provisional Patent Application Ser. No. 63/063,640, filed on Aug. 10, 2020, the entire disclosure of which is incorporated by reference herein.

### TECHNICAL FIELD

The disclosure relates to a minimally invasive surgical instrument and, more particularly, to an endoscope cleaning device for removing debris from a lens of an endoscope.

### BACKGROUND

Minimally invasive surgery eliminates the need to make a large incision in a patient, thereby reducing discomfort, recovery time, and many of the deleterious side effects associated with traditional open surgery. Minimally invasive viewing instruments such as, e.g., laparoscopes and endoscopes, provide viewing of internal tissues and/or organs during the minimally invasive surgery.

Laparoscopic surgery involves the placement of a laparoscope in a small incision in the abdominal wall of a patient, to view the surgical site. Endoscopic surgery involves the placement of an endoscope in a naturally occurring orifice, e.g., mouth, nose, anus, urethra, or vagina, to view the surgical site. Other minimally invasive surgical procedures include video assisted thoracic surgery and cardiovascular surgery conducted through small incisions between the ribs. These procedures also utilize scopes to view the surgical site.

A typical minimally invasive viewing instrument, e.g., a laparoscope or an endoscope, includes a housing, an elongated lens shaft extending from one end of the housing, and a lens that is provided in a distal end of the elongated lens shaft. A camera viewfinder extends from the other end of the housing. A camera is connected to the housing and transmits images sighted through the lens to an external monitor on which the images are displayed. During a surgical procedure, the distal end portion of the elongated lens shaft is extended into the patient, while the proximal end portion of the elongated lens shaft, the housing and the camera viewfinder remain outside the patient. In this manner, the laparoscope/endoscope is positioned and adjusted to view particular anatomical structures in the surgical field on the monitor.

During insertion of an endoscope or a laparoscope into the body and during the surgical procedure, debris, e.g., organic matter and/or moisture, may be deposited on the lens of the scope. The buildup of debris and condensation on the lens impairs visualization of the surgical site, and often necessitates cleaning of the lens.

### SUMMARY

The disclosure describes an endoscope cleaning device that demonstrates a practical approach to meeting the performance requirements and overcoming usability challenges associated with cleaning a lens of an endoscope during a surgical procedure.

In accordance with this disclosure, a surgical kit includes a surgical instrument including an elongate shaft and an end effector mounted on a distal end portion of the elongate shaft, an endoscope having a lens at a distal end portion of the endoscope, and a cleaning device. The cleaning device includes a sleeve defining a lumen dimensioned to receive the end effector and the elongate shaft therethrough, and an external part disposed on the sleeve to remove debris from the lens of the endoscope when the lens of the endoscope is wiped against the external part. The sleeve is flexible to be mountable about the elongate shaft of the surgical instrument. The external part defines a gap to collect debris on the lens of the endoscope.

In an aspect, the external part of the cleaning device may include a plurality of ribs on the sleeve. The plurality of ribs may define an acute angle with respect to a longitudinal axis defined by the sleeve.

In another aspect, the external part may be formed of an elastomer.

In yet another aspect, the sleeve may be formed of an elastomer.

In still yet another aspect, the cleaning device may be integrally formed as a single construct.

In an aspect, the cleaning device may be monolithically formed.

In another aspect, each rib of the plurality of ribs may be separate from other ribs.

In yet another aspect, the external part may be a rib having a continuous helical configuration.

In still yet another aspect, the external part may extend around a portion of a circumference of the sleeve.

In an aspect, the external part may be arranged along a length of the sleeve.

In another aspect, each ribs of the plurality of ribs may be tapered or beveled and may include a wiping portion configured to engage a surface of the lens of the endoscope to remove debris from the lens.

In yet another aspect, the external part may be a plurality of protuberances extending in a same general direction.

In an aspect, the plurality of protuberances may conform to the contour of the sleeve.

In another aspect, the plurality of protuberances may define a plane to engage a surface of the lens of the endoscope.

In yet another aspect, adjacent protuberances may define the gap therebetween to collect debris from the lens of the endoscope.

In still yet another aspect, the sleeve and the plurality of the protuberances may be formed of different materials.

In accordance with another aspect of the disclosure, a surgical kit includes a surgical instrument including an elongate shaft and an end effector mounted on a distal end portion of the elongate shaft, an endoscope having a lens at a distal end portion of the endoscope, and a cleaning device. The cleaning device includes a clamp body mountable on the elongate shaft of the surgical instrument, and an external part disposed on the clamp body to remove debris from the lens of the endoscope when the lens of the endoscope is wiped against the external part. The clamp body is flexible and defines a slot to receive the elongate shaft therethrough to secure the clamp body on the elongate shaft.

In an aspect, the external part may include a plurality of protuberances partially arranged around a circumference of the clamp body.

In accordance with another aspect of the disclosure, a surgical kit includes a surgical instrument including an elongate shaft and an end effector mounted on a distal end portion of the elongate shaft, and a cleaning device including a base and a plurality of protuberances extending from the base to remove debris from a lens of an endoscope when the lens of the endoscope is wiped against the plurality of protuberances. The base is transitionable between a first configuration, in which, the base defines a planar surface, and a second configuration, in which, the base conforms to an exterior surface of the elongate shaft of the surgical instrument to mount the base to the surgical instrument.

In an aspect, the plurality of protuberances may be formed of silicone or rubber.

### BRIEF DESCRIPTION OF DRAWINGS

The above and other aspects and features of this disclosure will become more apparent in view of the following detailed description when taken in conjunction with the accompanying drawings wherein like reference numerals identify similar or identical elements.

FIG. 1 is a perspective view of a cleaning device in accordance with the disclosure, illustrating the cleaning device mounted on an elongate shaft of a surgical instrument;

FIG. 2 is a perspective view of the cleaning device of FIG. 1, illustrating the cleaning device separated from the elongate shaft of the surgical instrument;

FIG. 3A is a perspective view of the cleaning device of FIG. 1;

FIG. 3B is a side view of the cleaning device of FIG. 3A;

FIG. 3C is a cross-sectional view of the cleaning device of FIG. 3B;

FIG. 3D is a perspective view of a cleaning device in accordance with another aspect of the disclosure;

FIG. 3E is a side view of the cleaning device of FIG. 3D;

FIG. 3F is a cross-sectional view of the cleaning device of FIG. 3D;

FIG. 4 is a perspective view of the cleaning device of FIG. 1, illustrating removal of debris from a lens of an endoscope;

FIG. 5A is a perspective view of a cleaning device in accordance with another aspect of the disclosure;

FIG. 5B is a side view of the cleaning device of FIG. 5A;

FIG. 5C is a top view of the cleaning device of FIG. 5A;

FIG. 5D is a bottom view of the cleaning device of FIG. 5A;

FIG. 5E is a cross-sectional view of the cleaning device of FIG. 5A;

FIG. 6A is a perspective view of a cleaning device in accordance with another aspect of the disclosure;

FIG. 6B is a side view of the cleaning device of FIG. 6A;

FIG. 6C is a top view of the cleaning device of FIG. 6A;

FIG. 6D is a bottom view of the cleaning device of FIG. 6A;

FIG. 6E is a cross-sectional view of the cleaning device of FIG. 6A;

FIG. 7A is a perspective view of a cleaning device in accordance with another aspect of the disclosure;

FIG. 7B is a side view of the cleaning device of FIG. 7A;

FIG. 7C is a cross-sectional view of the cleaning device of FIG. 7A;

FIG. 8A is a perspective view of a cleaning device in accordance with another aspect of the disclosure;

FIG. 8B is a side view of the cleaning device of FIG. 8A;

FIG. 8C is a top view of the cleaning device of FIG. 8A;

FIG. 8D is a bottom view of the cleaning device of FIG. 8A;

FIG. 8E is a cross-sectional view of the cleaning device of FIG. 8A;

FIG. 9A is a perspective view of a cleaning device in accordance with another aspect of the disclosure;

FIG. 9B is a side view of the cleaning device of FIG. 9A;

FIG. 9C is a top view of the cleaning device of FIG. 9A;

FIG. 9D is a bottom view of the cleaning device of FIG. 9A;

FIG. 9E is a cross-sectional view of the cleaning device of FIG. 9A;

FIG. 10A is a perspective view of a cleaning device in accordance with another aspect of the disclosure;

FIG. 10B is a side view of the cleaning device of FIG. 10A;

FIG. 10C a cross-sectional view of the cleaning device of FIG. 10A;

FIG. 11A is a perspective view of a cleaning device in accordance with another aspect of the disclosure;

FIG. 11B is a side view of the cleaning device of FIG. 11A;

FIG. 11C is a top view of the cleaning device of FIG. 11A;

FIG. 11D is a bottom view of the cleaning device of FIG. 11A;

FIG. 11E is a cross-sectional view of the cleaning device of FIG. 11A;

FIG. 12A is a perspective view of a cleaning device in accordance with another aspect of the disclosure;

FIG. 12B is a side view of the cleaning device of FIG. 12A;

FIG. 12C is a top view of the cleaning device of FIG. 12A;

FIG. 12D is a bottom view of the cleaning device of FIG. 12A;

FIG. 12E is a cross-sectional view of the cleaning device of FIG. 12A;

FIG. 12F is a perspective view of a cleaning device in accordance with another aspect of the disclosure;

FIG. 12G is an exploded perspective view of the cleaning device of FIG. 12F with parts separated;

FIG. 13A is a perspective view of a cleaning device in accordance with another aspect of the disclosure;

FIG. 13B is a side view of the cleaning device of FIG. 13A;

FIG. 13C is a cross-sectional view of the cleaning device of FIG. 13A;

FIG. 14 is a perspective view of a cleaning device in accordance with another aspect of the disclosure, illustrating use with the surgical instrument of FIG. 1;

FIG. 15 is a perspective view of the cleaning device of FIG. 14, illustrating the cleaning device separated from the surgical instrument of FIG. 1;

FIG. 16 is a perspective view of the cleaning device of FIG. 14;

FIG. 17 is a side view of the cleaning device of FIG. 16;

FIG. 18 is a top view of the cleaning device of FIG. 16;

FIG. 19 is a bottom view of the cleaning device of FIG. 16;

FIG. 20 is a cross-sectional view of the cleaning device of FIG. 16;

FIG. 21 is a perspective view of a cleaning device in accordance with another aspect of the disclosure, illustrating use with the surgical instrument of FIG. 1;

FIG. 22 is an exploded perspective view of the cleaning device of FIG. 21 with parts separated, illustrating use with the surgical instrument of FIG. 1;

FIG. 23 is an end view of a cleaning device in accordance with yet another aspect of the disclosure; and

FIG. 24 is an end view of the cleaning device of FIG. 23, illustrating the cleaning device mounted on the surgical instrument of FIG. 1.

### DETAILED DESCRIPTION

The endoscope cleaning device disclosed herein is described in detail with reference to the drawings, in which like reference numerals designate identical or corresponding elements in each of the several views.

As used herein, the term “distal” refers to the portion that is being described which is farther from a user, while the term “proximal” refers to the portion that is being described which is closer to a user. In addition, the terms parallel and perpendicular are understood to include relative configurations that are substantially parallel and substantially perpendicular up to about + or −10 degrees from true parallel and true perpendicular. Further, to the extent consistent, any or all of the aspects detailed herein may be used in conjunction with any or all of the other aspects detailed herein.

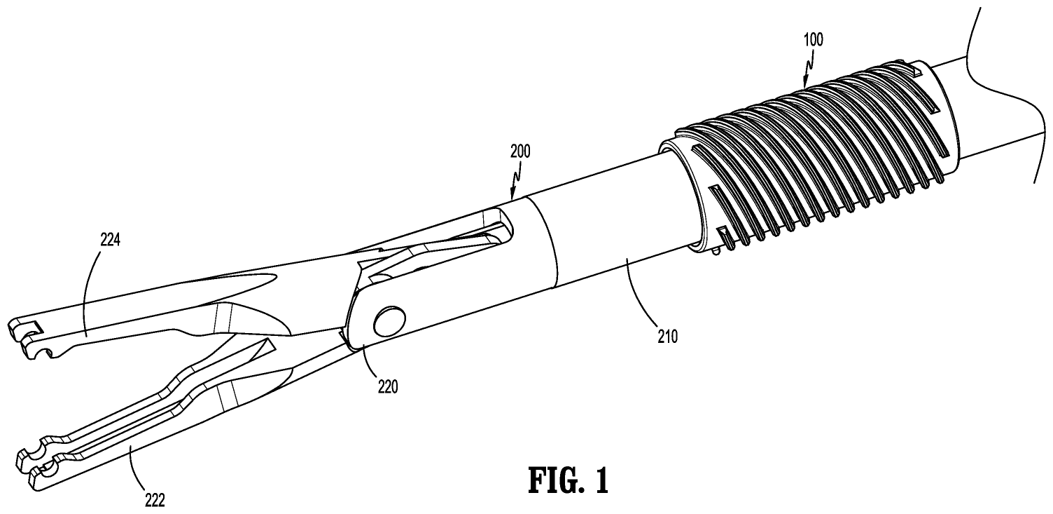

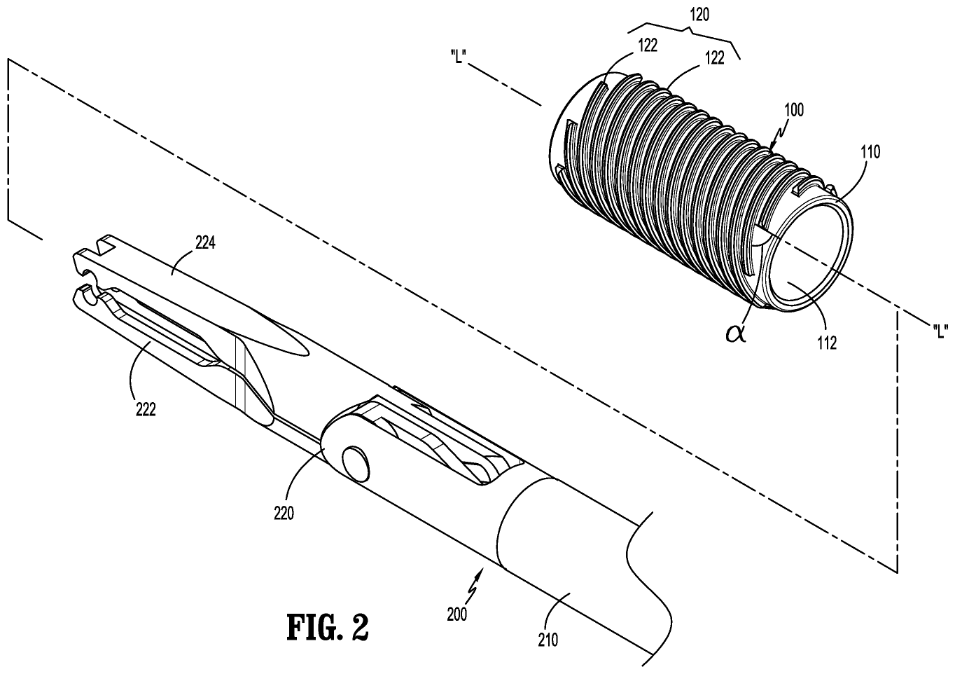

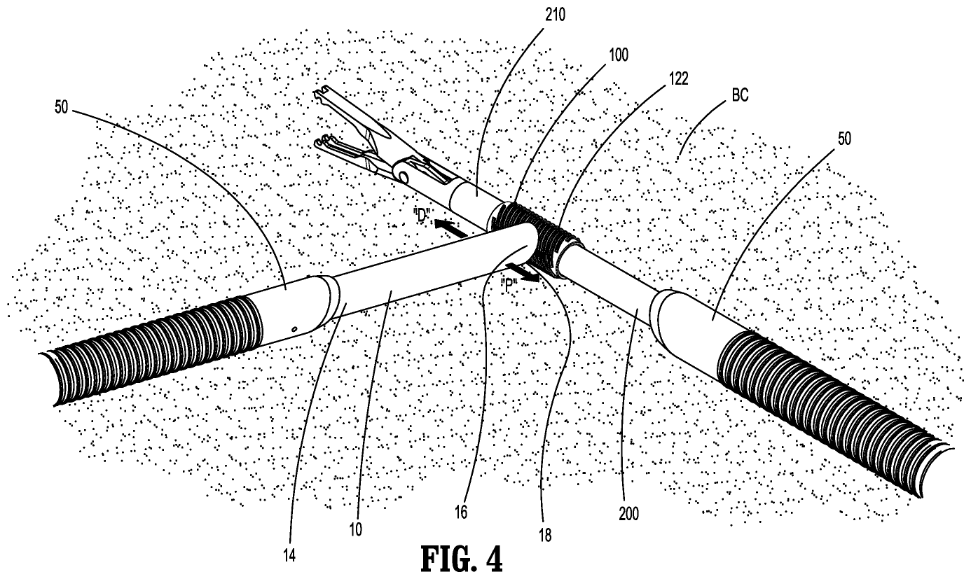

In FIGS. 1 and 2, an exemplary in situ lens cleaning device in accordance with the disclosure is shown generally as a cleaning device 100. The cleaning device 100 enables cleaning of a lens 18 (FIG. 4) of an endoscope 10 during a surgical procedure to maintain a clear image without having to remove the endoscope 10 from the patient's body. In particular, the lens 18 may be wiped against the cleaning device 100 to remove debris such as, e.g., organic matter and/or moisture, therefrom. Further, the cleaning device 100 does not require additional modification to a minimally invasive surgical instrument for use therewith, as will be discussed below. FIG. 1 illustrates the cleaning device 100 being used with a minimally invasive surgical instrument in the form of, e.g., a surgical clip applier 200, having an elongate shaft 210 and an end effector 220 coupled to a distal end portion of the elongate shaft 210. It is contemplated that the minimally invasive surgical instrument may be a surgical instrument such as laparoscopic or endoscopic graspers, obturators, dissectors, retractors, staplers, laser probes, photographic devices, tubes, endoscopes and laparoscopes, electro-surgical devices and the like.

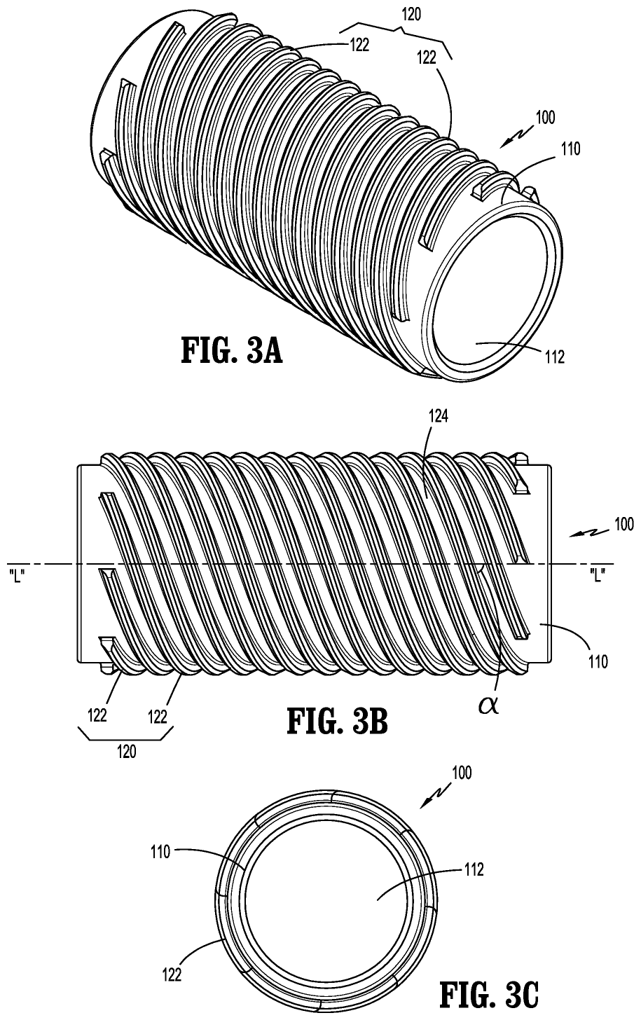

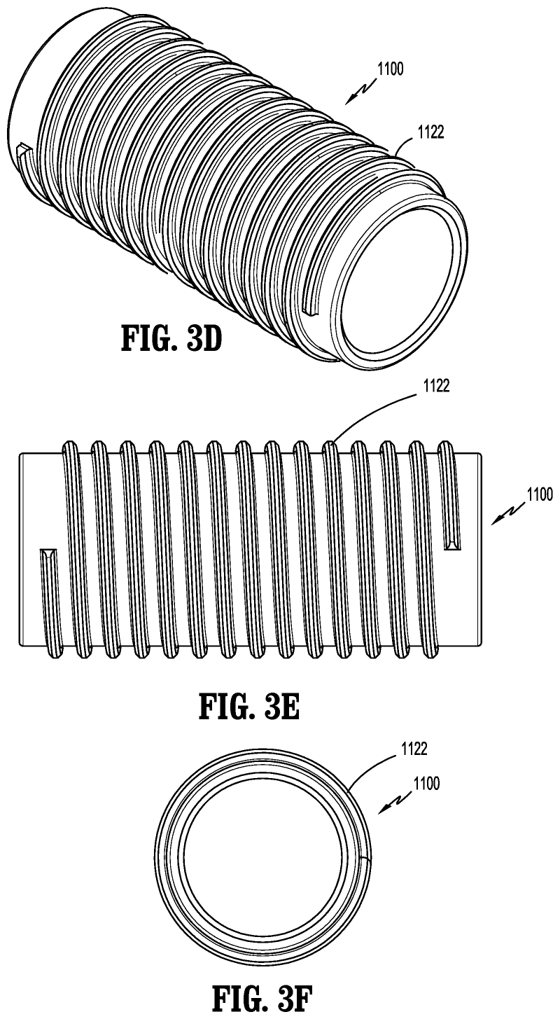

The end effector 220 of the surgical clip applier 200 includes first and second jaws 222, 224 configured to support clips therebetween and apply the clips to, e.g., tissue or a surgical object. FIGS. 3A-3C illustrate the cleaning device 100 that is attachable to the elongate shaft 210 (FIG. 2) of the surgical clip applier 200. The cleaning device 100 includes a sleeve or a body 110 having a tubular configuration. The sleeve defines a lumen 112 dimensioned to receive the end effector 220 and the elongate shaft 210 therethrough (FIG. 1). The sleeve 110 may be formed of a flexible or a resilient material such as, e.g., an elastomer. Under such a configuration, the sleeve 110 may be frictionally mounted on various surgical instruments having different dimensions. In this manner, the cleaning device 100 is also detachable from the elongate shaft 210 of the surgical clip applier 200. However, it is contemplated that the sleeve 110 may be secured to the elongate shaft 210 through the use of, e.g., ultrasonic welding, glue, and snap-fit. The cleaning device 100 further includes an external part 120 that enables removal of debris, e.g., organic matter and/or moisture, from the lens 18 (FIG. 4) of the endoscope 10 when the clinician wipes the lens 18 against the external part 120, as will be described hereinbelow.

The external part 120 includes a plurality of ribs 122 extending from the sleeve 110. The plurality of ribs 122 is arranged along a length of the sleeve 110. The plurality of ribs 122 is circumferentially arranged about a longitudinal axis “L-L” defined by the cleaning device 100. In particular, each rib 122 may define, e.g., an acute, angle α with respect to the longitudinal axis “L-L” to enhance removal of debris from the lens 18 (FIG. 4) of the endoscope 10. In addition, each rib 122 may be discrete and spaced from an adjacent rib 122 to further enhance removal of debris. The plurality of ribs 122 has a helical configuration with starting points and ending points that are longitudinally staggered. Adjacent ribs 122 may define a gap 124 therebetween to collect and/or retain debris from the lens 18. The plurality of ribs 122 may be formed of an elastomer such as, e.g., rubber or silicone. In addition, the plurality of ribs 122 and the sleeve 110 may be integrally formed, and alternatively, monolithically formed. It is contemplated that the sleeve 110 may define a non-circular cross-section such as, e.g., triangular cross-section. It is contemplated that the external part 120 may be a single continuous rib structure having a continuous helical configuration. For example, FIGS. 3D-3F illustrate a cleaning device 1100 having a single continuous rib 1122. While the cleaning device 100 is shown as a detachable component separate from the elongate shaft 210 of the surgical clip applier 200, it is contemplated that the cleaning device 100 may be integrally formed with the surgical clip applier 200 as a single construct. For example, the cleaning device 100 may be over-molded onto the elongate shaft 210 of the surgical clip applier 200.

FIG. 4 illustrates the use of the cleaning device 100 with the endoscope 10. In particular, the endoscope 10 includes a housing and an elongated tubular shaft 14 extending distally from the housing and terminating in the lens 18. A distal tip portion 16 of the endoscope 10 includes a number of optical components that produce images of the patient's tissues as known by one skilled in the art. The optical components generally include a window or front element of a lens assembly that is positioned in front of an image sensor or in front of a fiber optic imaging guide that transfers an image to the proximal end of the endoscope 10. Illumination sources such as, e.g., light-emitting diodes, fiber optic or illumination guides, may also be provided. The elongate tubular shaft 14 may be rigid, semi-rigid, or flexible. The housing may include a viewfinder adapted to sight images of a surgical field in the patient, e.g. an abdominal cavity, thoracic cavity, etc., as the position of the endoscope 10 is adjusted to view a particular anatomical structure in the surgical field. A camera is adapted to receive images of the surgical field sighted through the lens 18 and transmit the images to, e.g., an external monitor, on which the images of the surgical field are displayed. That is, a visual display device converts the optical signal into a video signal to produce a video image on the monitor (or for storage on select media). Accordingly, the monitor enables a clinician to view the anatomical structure in the surgical field inside the patient as the surgical procedure is carried out using minimally invasive or endoscopic surgical instruments. Throughout the surgical procedure, condensation, smoke particles, and biological tissue or matter have a tendency to contact and build up on the lens 18 of the endoscope 10. This tends to obscure the images of the surgical field as they are displayed on the monitor. To this end, the cleaning device 100 that may be utilized during the surgical procedure to maintain a clear image without having to remove the endoscope 10 from the patient's body, as will be discussed hereinbelow.

Prior to use, the cleaning device 100 may be mounted on the elongate shaft 210 of the surgical clip applier 200 by sliding the cleaning device 100 over the end effector 220 and onto the elongate shaft 210. The clinician may utilize one or more cannulas 50. The cannulas 50 are configured to permit access to a surgical site such as, e.g., an insufflated abdominal cavity, during a laparoscopic procedure to permit the introduction of a surgical instrument such as the surgical clip applier 200 for performing various surgical tasks on internal organs within the cavity.

Initially, the surgical clip applier 200 is inserted into a body cavity “BC” through the cannula 50, and the endoscope 10 is inserted into the body cavity “BC” through a second cannula 50. The surgical clip applier 200 may be utilized as needed by the clinician and the lens 18 of the endoscope 10 may be manipulated as needed in order to be placed adjacent the surgical site to provide a visual aid to the clinician. When the lens 18 of the endoscope 10 is obscured by debris, e.g., organic matter and/or moisture, the clinician may place the lens 18 of the endoscope 10 on the plurality of ribs 122 of the cleaning device 100. For example, the endoscope 10 may be transversely arranged with respect to the surgical clip applier 200. The clinician may manipulate the endoscope 10 to move the lens 18 in the direction of arrows “P” and “D” with the surface of the lens 18 in contact with one or more of the ribs 122 of the plurality of ribs 122, during which time, the plurality of ribs 122 removes debris from the lens 18 of the endoscope 10. For example, the debris may be captured in the gap 124 (FIG. 3B) between adjacent ribs 122. In this manner, the lens 18 of the endoscope 10 may be cleaned without removing the lens 18 from the body cavity “BC.” The cleaning of the lens 18 may be repeated as needed during the surgical procedure. It is also envisioned that the cleaning device 100 may be adapted for use with a robotic surgical system.

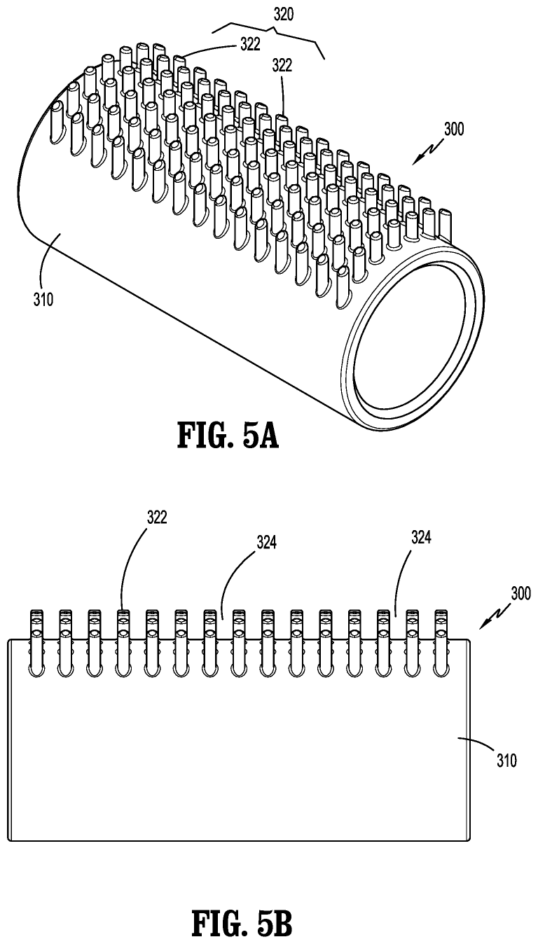

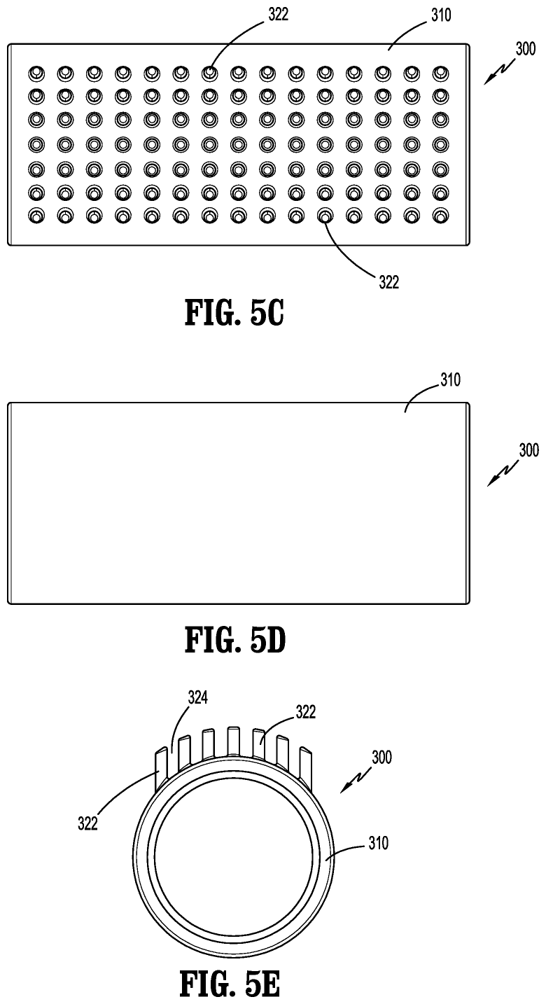

FIGS. 5A-5E illustrate a cleaning device 300 in accordance with another aspect of the disclosure. In the interest of brevity, portions of the cleaning device 300 substantially similar to corresponding portions of the cleaning devices 100, 1100 will not be described herein to avoid obscuring the disclosure in unnecessary detail. The cleaning device 300 includes a sleeve 310 having a tubular configuration and an external part 320 including a plurality of protuberances 322 extending outwardly from the sleeve 310 in the same general direction (see, e.g., FIG. 5E). The plurality of protuberances 322 may have, e.g., a same length, such that the plurality of protuberances 322 conform to a curvature of the sleeve 310. The plurality of protuberances 322 is arranged along a length of the cleaning device 300. The plurality of protuberances 322 is partially arranged around a circumference of the sleeve 310. Adjacent protuberances 322 may define, e.g., a uniform, gap 324 to receive and retain debris from the lens 18 (FIG. 4) of the endoscope 10. The plurality of protuberances 322 and the sleeve 310 may be integrally formed, or alternatively, monolithically formed of an elastomer such as, e.g., rubber or silicone.

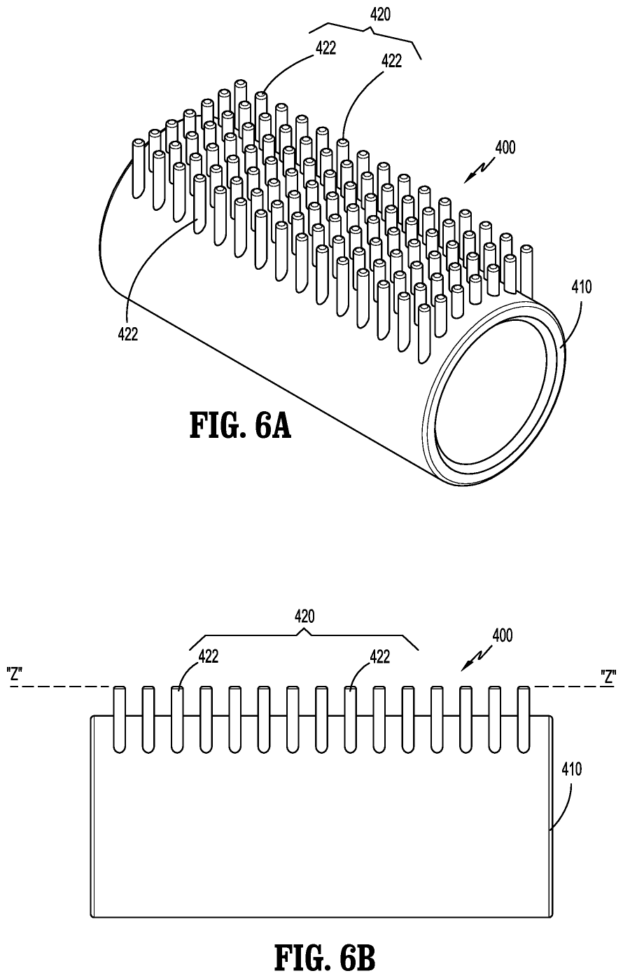

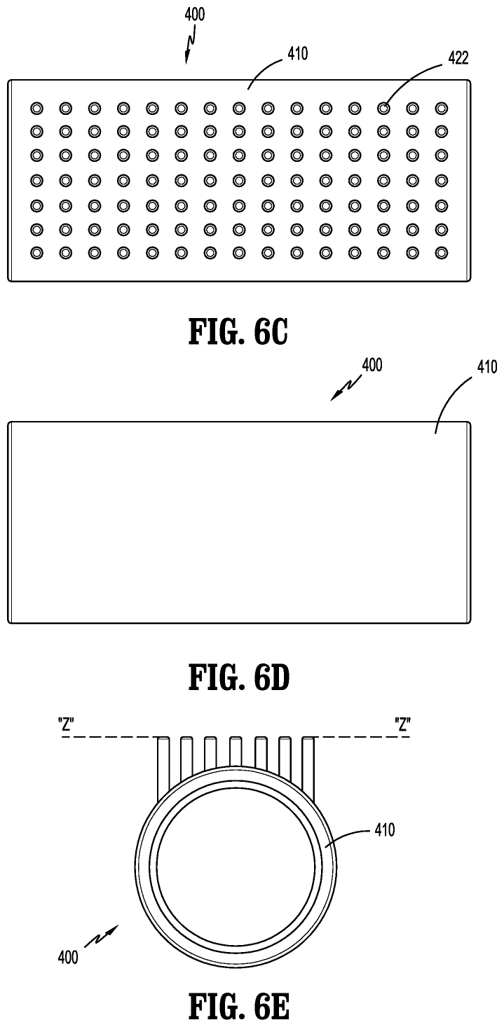

FIGS. 6A-6E illustrate a cleaning device 400 in accordance with another aspect of the disclosure. In the interest of brevity, portions of the cleaning device 400 substantially similar to corresponding portions of the cleaning devices 100, 300, 1100 will not be described herein to avoid obscuring the disclosure in unnecessary detail. The cleaning device 400 includes a sleeve 410 having a tubular configuration and an external part 420 including a plurality of protuberances 422 extending outwardly from the sleeve 410 in the same general direction (see, e.g., FIG. 6E). However, in contrast to the plurality of protuberances 322 of the cleaning device 300, the plurality of protuberances 422 has varying lengths such that the plurality of protuberances 422 defines a plane “Z” that may provide enhanced contact with the lens 18 having a planar surface. The plurality of protuberances 422 and the sleeve 410 may be integrally formed, or alternatively, monolithically formed of an elastomer such as, e.g., rubber or silicone.

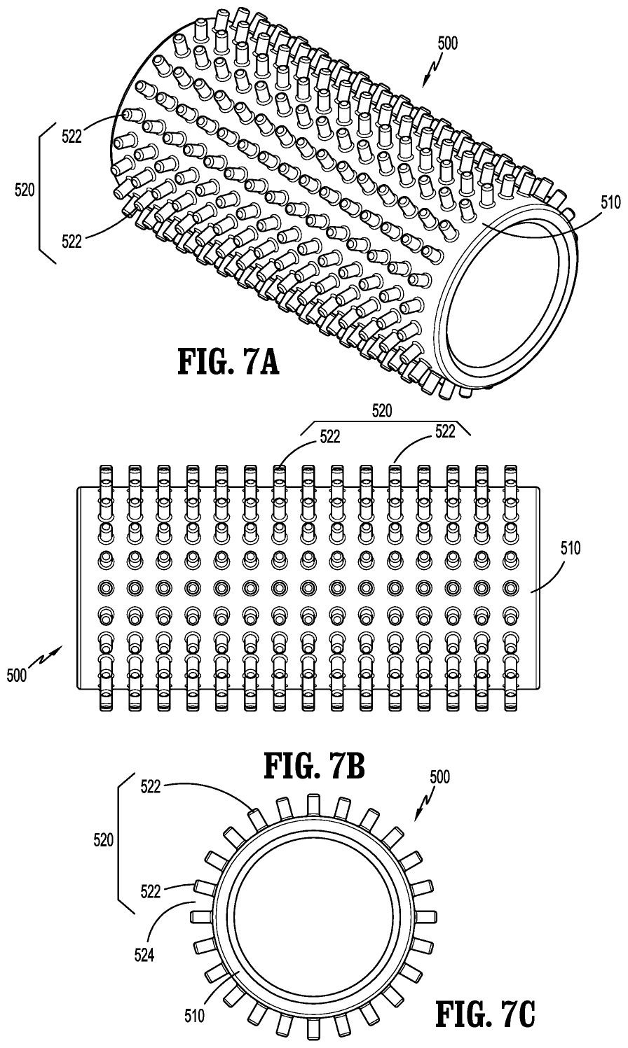

FIGS. 7A-7C illustrate a cleaning device 500 in accordance with another aspect of the disclosure. In the interest of brevity, portions of the cleaning device 500 substantially similar to corresponding portions of the cleaning devices 100, 300, 400, 1100 will not be described herein to avoid obscuring the disclosure in unnecessary detail. The cleaning device 500 includes a sleeve 510 having a tubular configuration and an external part 520 including a plurality of protuberances 522 that extends outwardly from the sleeve 510. Each protuberance 522 of the plurality of protuberances 522 is orthogonal to the sleeve 510. The plurality of protuberances 322 may have a uniform radial length such that the plurality of protuberances 522 conforms to a curvature of the sleeve 510. The plurality of protuberances 522 is arranged along a length of the cleaning device 500. The plurality of protuberances 522 is circumferentially arranged about the sleeve 510.

Adjacent protuberances 522 may define, e.g., a uniform, gap 524 to receive and retain debris from the lens 18 (FIG. 4) of the endoscope 10. The plurality of protuberances 522 and the sleeve 510 may be integrally formed, or alternatively, monolithically formed of an elastomer such as, e.g., rubber or silicone.

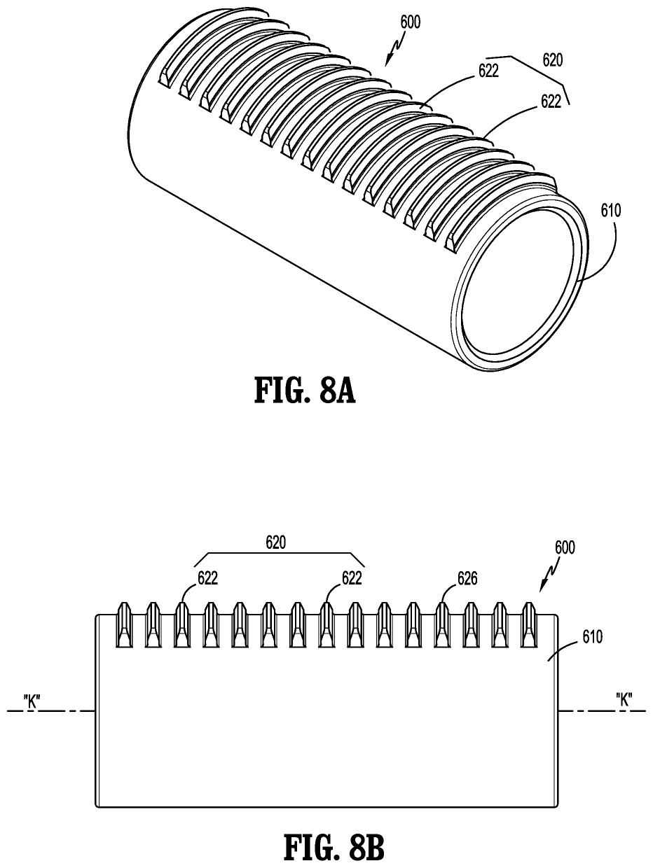

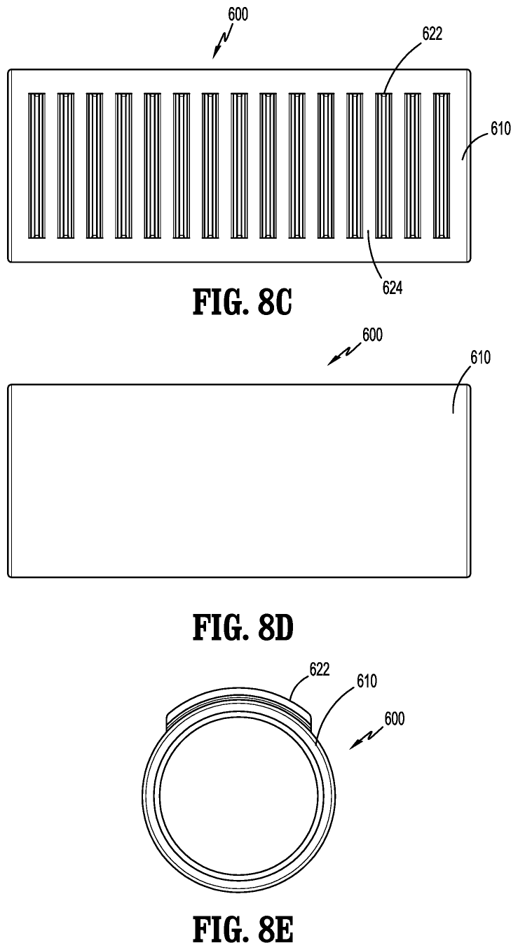

FIGS. 8A-8E illustrate a cleaning device 600 in accordance with another aspect of the disclosure. In the interest of brevity, portions of the cleaning device 600 substantially similar to corresponding portions of the cleaning devices 100, 300, 400, 500, 1100 will not be described herein to avoid obscuring the disclosure in unnecessary detail. The cleaning device 600 includes a sleeve 610 having a tubular configuration and an external part 620 including a plurality of ribs 622 protruding from the sleeve 610. Each rib 622 extends partially around a circumference of the sleeve 610. The plurality of ribs 622 is arranged along a length of the sleeve 610. Each rib 622 is tapered or beveled and has an engaging portion 626 to wipe the lens 18 (FIG. 4) of the endoscope 10 to remove debris from the lens 18. The plurality of ribs 622 has a same thickness (extending from the sleeve 610) such that the plurality of ribs 622 conforms to a curvature of the sleeve 610. Adjacent ribs 622 may define, e.g., a uniform, gap 624 to receive and retain debris from the lens 18 (FIG. 4) of the endoscope 10. The plurality of ribs 622 and the sleeve 610 may be integrally formed, or alternatively, monolithically formed of an elastomer such as, e.g., rubber or silicone.

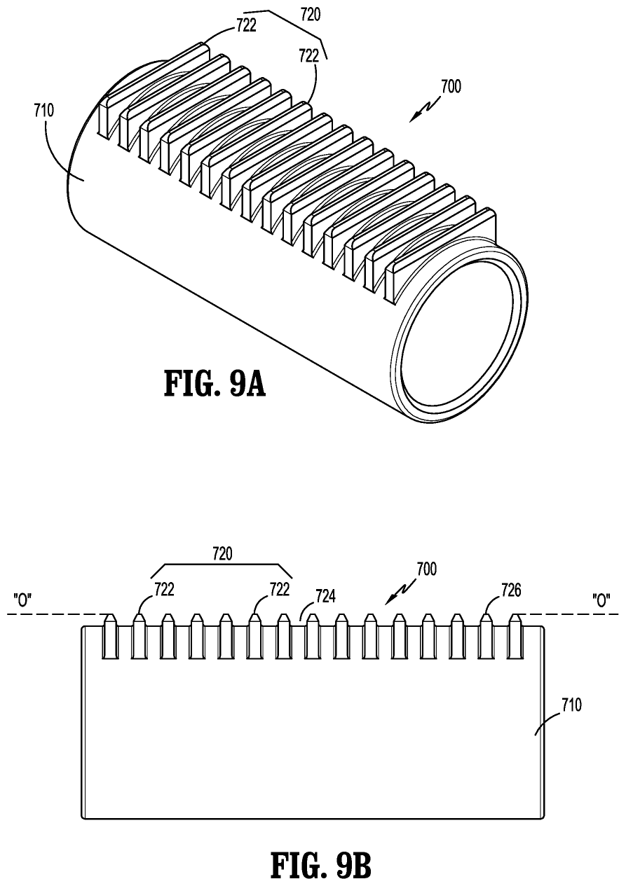

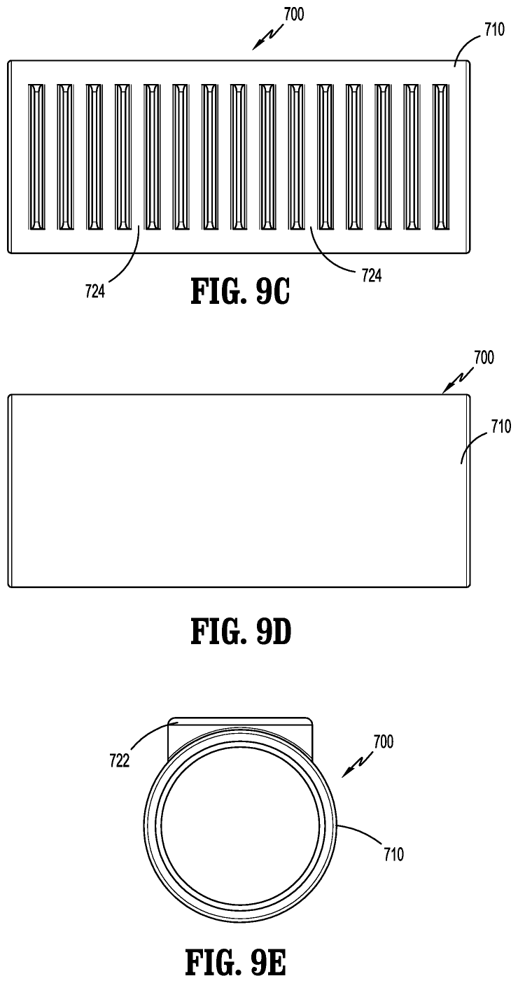

FIGS. 9A-9E illustrate a cleaning device 700 in accordance with another aspect of the disclosure. In the interest of brevity, portions of the cleaning device 700 substantially similar to corresponding portions of the cleaning devices 100, 300, 400, 500, 600, 1100 will not be described herein to avoid obscuring the disclosure in unnecessary detail. The cleaning device 700 includes a sleeve 710 having a tubular configuration and an external part 720 including a plurality of ribs 722 protruding from the sleeve 710. Each rib 722 extends orthogonally from the sleeve 710 and partially around a circumference of the sleeve 710. The plurality of ribs 722 are arranged along a length of the sleeve 710. Each rib 722 is tapered or beveled and has an engaging portion 726 configured to wipe the lens 18 (FIG. 4) of the endoscope 10 to remove debris from the lens 18. The plurality of ribs 722 extends from the sleeve 710 in a manner that defines a plane “0” to engage, e.g., a planar surface, of the lens 18. Adjacent ribs 722 may define, e.g., a uniform, gap 724 to receive and retain debris from the lens 18 (FIG. 4) of the endoscope 10. The plurality of ribs 722 and the sleeve 710 may be integrally formed, or alternatively, monolithically formed of an elastomer such as, e.g., rubber or silicone.

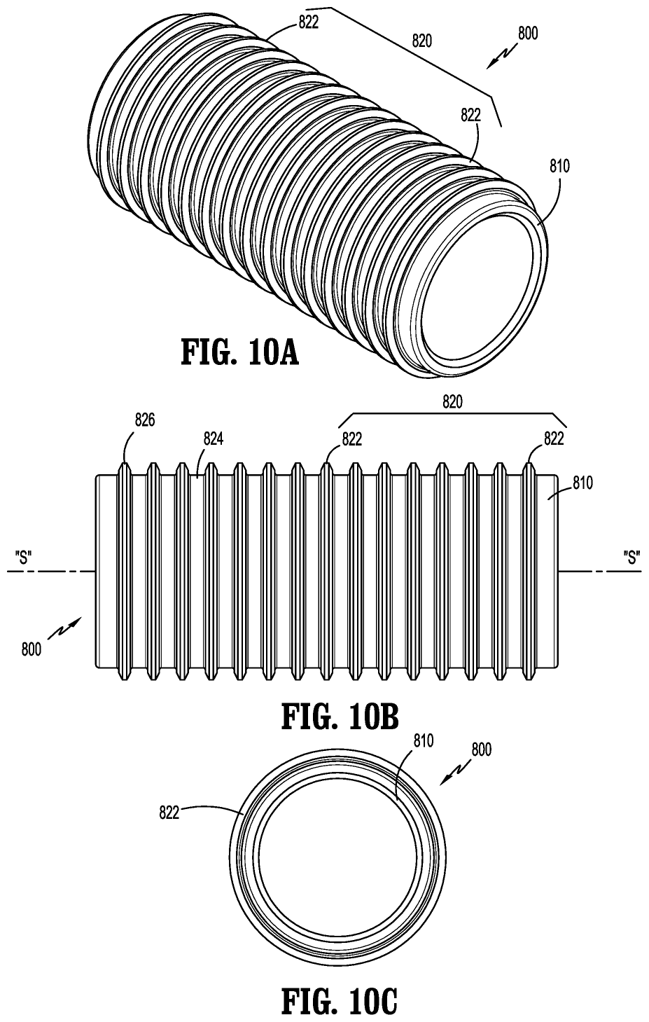

FIGS. 10A-10C illustrate a cleaning device 800 in accordance with another aspect of the disclosure. In the interest of brevity, portions of the cleaning device 800 substantially similar to corresponding portions of the cleaning devices 100, 300, 400, 500, 600, 700, 1100 will not be described herein to avoid obscuring the disclosure in unnecessary detail. The cleaning device 800 includes a sleeve 810 having a tubular configuration and an external part 820 including a plurality of ribs 822 protruding from the sleeve 810. Each rib 822 extends circumferentially about the sleeve 810. Each rib 822 may be orthogonal to a longitudinal axis “S-S” defined by the sleeve 810. The plurality of ribs 822 is arranged along a length of the sleeve 810. As discussed hereinabove, each rib 822 may be tapered or beveled and may have an engaging portion 826 to wipe the lens 18 (FIG. 4) of the endoscope 10 to remove debris from the lens 18. The plurality of ribs 822 has a uniform thickness extending from the sleeve 810. Adjacent ribs 822 may define, e.g., a uniform, gap 824 to receive and retain debris from the lens 18 (FIG. 4) of the endoscope 10. The plurality of ribs 822 and the sleeve 810 may be integrally formed, or alternatively, monolithically formed of an elastomer such as, e.g., rubber or silicone.

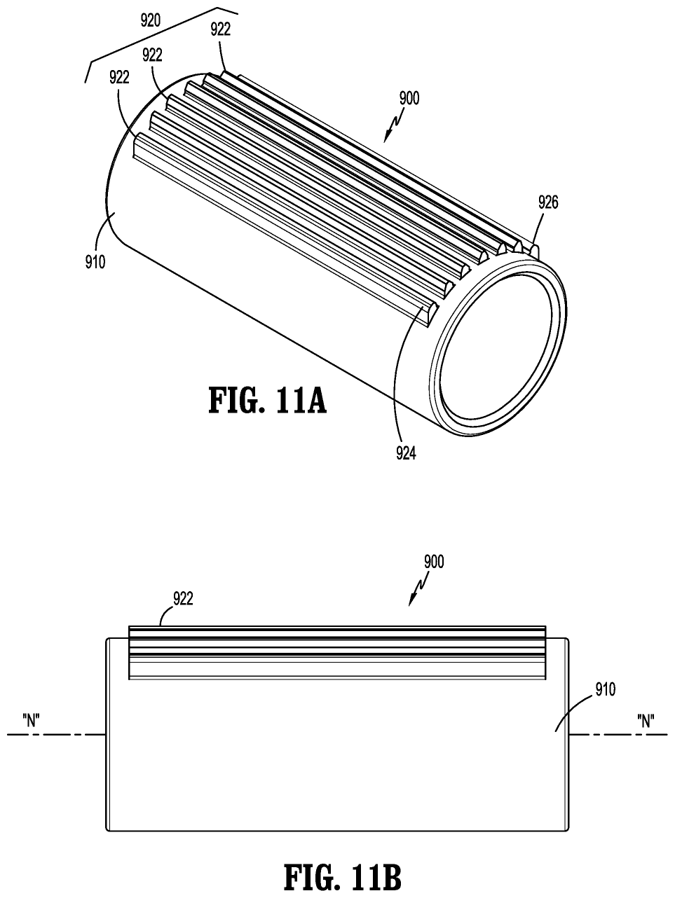

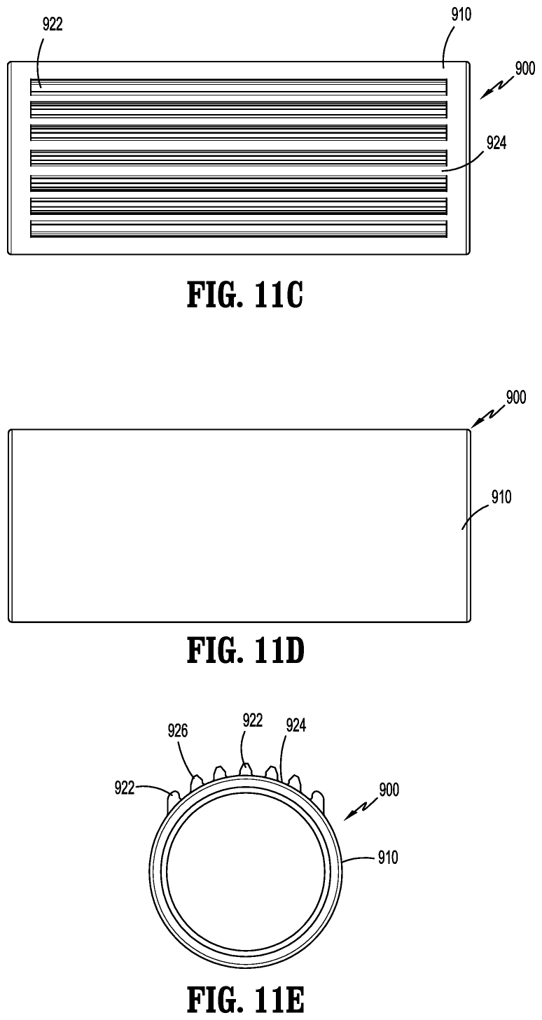

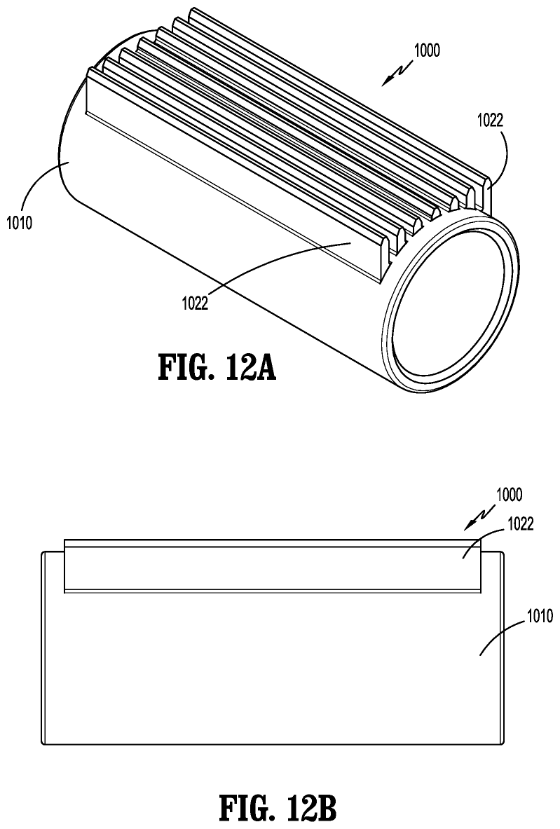

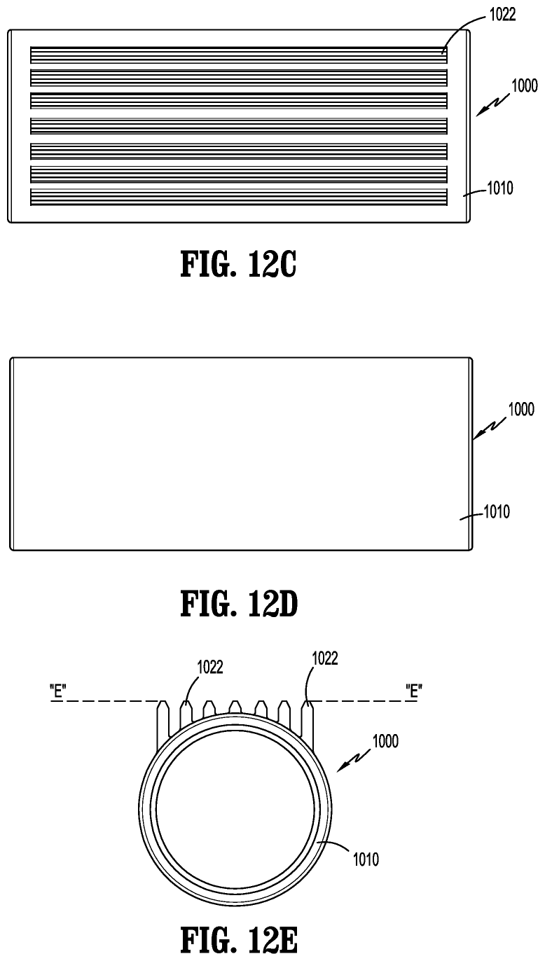

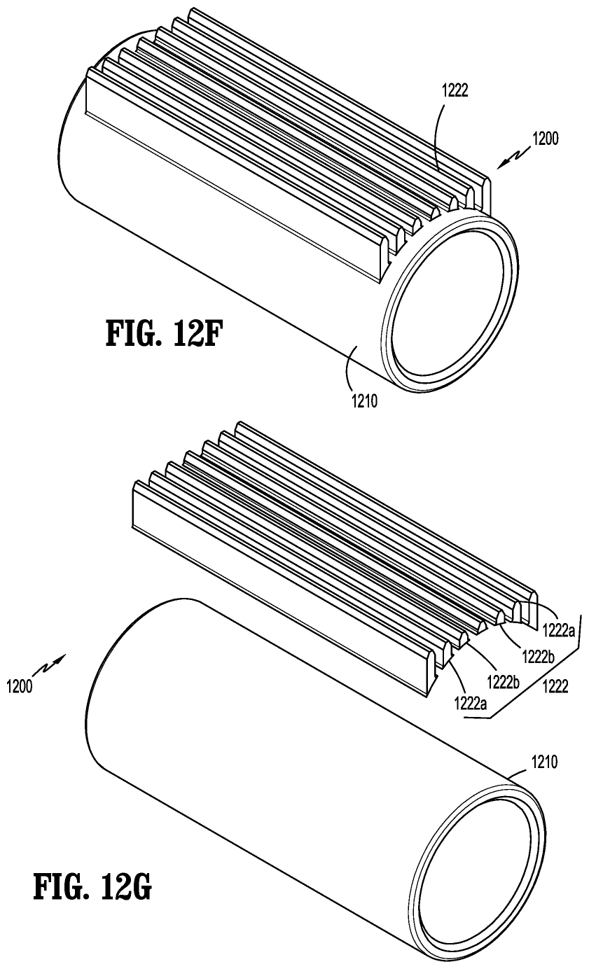

FIGS. 11A-11E illustrate a cleaning device 900 in accordance with another aspect of the disclosure. In the interest of brevity, portions of the cleaning device 900 substantially similar to corresponding portions of the cleaning devices 100, 300, 400, 500, 600, 700, 800, 1100 will not be described herein to avoid obscuring the disclosure in unnecessary detail. The cleaning device 900 includes a sleeve 910 having a tubular configuration and an external part 920 including a plurality of ribs 922 protruding from the sleeve 910. The plurality of the ribs 922 is partially arranged around a circumference of the sleeve 910. Each rib 922 extends along a length of the sleeve 910. Adjacent ribs 922 may define, e.g., a uniform, gap 924 to receive and retain debris from the lens 18 (FIG. 4) of the endoscope 10. Each gap 924 runs parallel to a longitudinal axis “N-N” defined by the sleeve 910. Further, each rib 922 may be tapered or beveled and may have an engaging portion 926 to wipe the lens 18 of the endoscope 10 to remove debris from the lens 18. The plurality of ribs 922 may have, e.g., non-uniform, thickness extending from the sleeve 910 such that the plurality of ribs 922 conform to the curvature of the sleeve 910. However, it is contemplated that a cleaning device 1000 (FIGS. 12A-12E) may include a plurality of ribs 1022 having varying thicknesses extending from a sleeve 1010 of the cleaning device 1000 such that the plurality of ribs 1022 define a plane “E” to engage the lens 18. The plurality of ribs 922 and the sleeve 910 may be integrally formed, or alternatively, monolithically formed of an elastomer such as, e.g., rubber or silicone. In another aspect, FIGS. 12F and 12G illustrate a cleaning device 1200 including the sleeve 1210 and a plurality of ribs 1222 formed of different materials. In addition, the plurality of ribs 1222 may be separate from the sleeve 1210 such that the plurality of ribs 1222 may be, e.g., over-molded, on the sleeve 1210. It is also contemplated that the plurality of ribs 1222 may include first ribs 1222a formed of a first material and second ribs 1222b formed of a second material different from the first ribs 1222a.

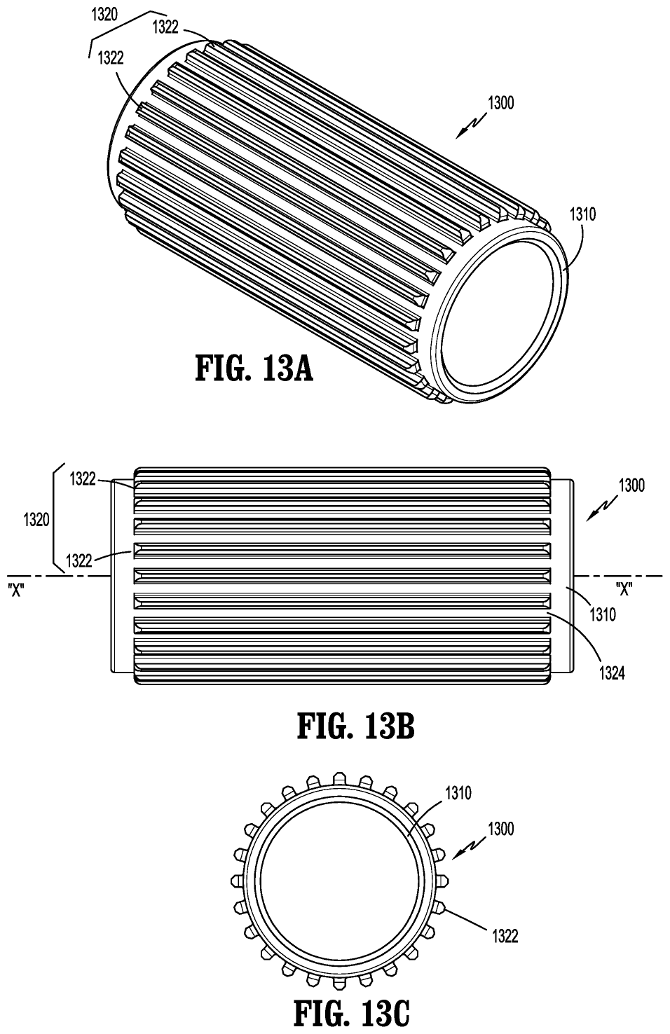

FIGS. 13A-13C illustrate a cleaning device 1300 in accordance with another aspect of the disclosure. In the interest of brevity, portions of the cleaning device 1300 substantially similar to corresponding portions of the cleaning devices 100, 300, 400, 500, 600, 700, 800, 900, 1000, 1100, 1200 will not be described herein to avoid obscuring the disclosure in unnecessary detail. The cleaning device 1300 includes a sleeve 1310 having a tubular configuration and an external part 1320 including a plurality of ribs 1322 extending outwardly from the sleeve 1310. The plurality of ribs 1322 extends axially along a length of sleeve 1310 (i.e., parallel to a longitudinal axis “X-X” defined by the sleeve 310). The plurality of ribs 1322 may have uniform lengths. In addition, the plurality of ribs 1322 is circumferentially arranged about the sleeve 1310. Further, the plurality of ribs 1322 has uniform thickness extending from the sleeve 1310 such that the plurality of ribs 1322 conforms to a curvature of the sleeve 1310. Adjacent ribs 1322 may define, e.g., a uniform, gap 1324 to receive and retain debris from the lens 18 (FIG. 4) of the endoscope 10. The plurality of ribs 1322 and the sleeve 1310 may be integrally formed, or alternatively, monolithically formed of an elastomer such as, e.g., rubber or silicone.

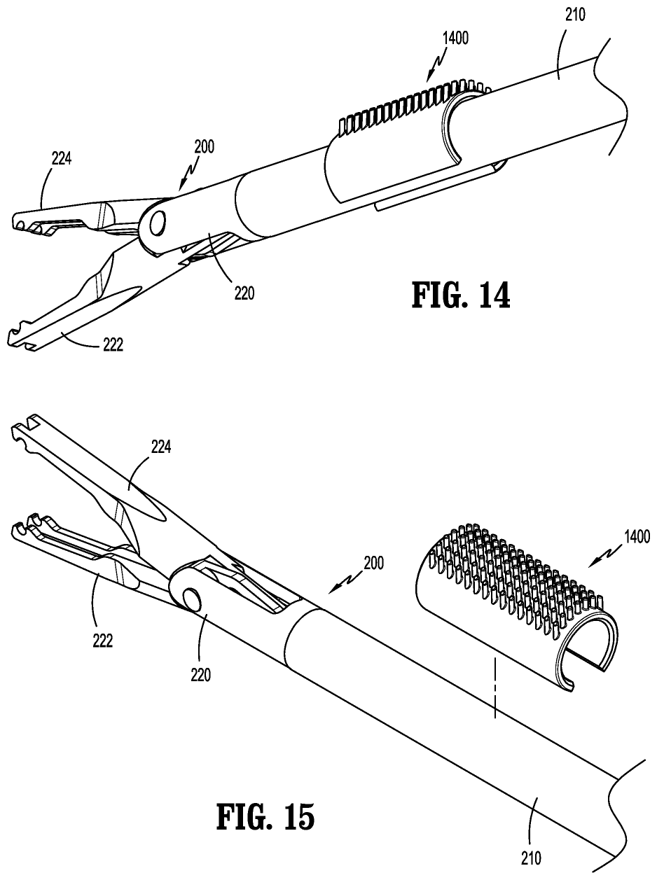

FIGS. 14 and 15 illustrate a cleaning device 1400 in accordance with another aspect of the disclosure. In the interest of brevity, portions of the cleaning device 1400 substantially similar to corresponding portions of the above-identified cleaning devices 100, 300, 400, 500, 600, 700, 800, 900, 1000, 1100, 1200, 1300 will not be described herein to avoid obscuring the disclosure in unnecessary detail. The cleaning device 1400 is configured to be detachably snapped onto the elongate shaft 210 of the surgical clip applier 200 as discussed hereinafter.

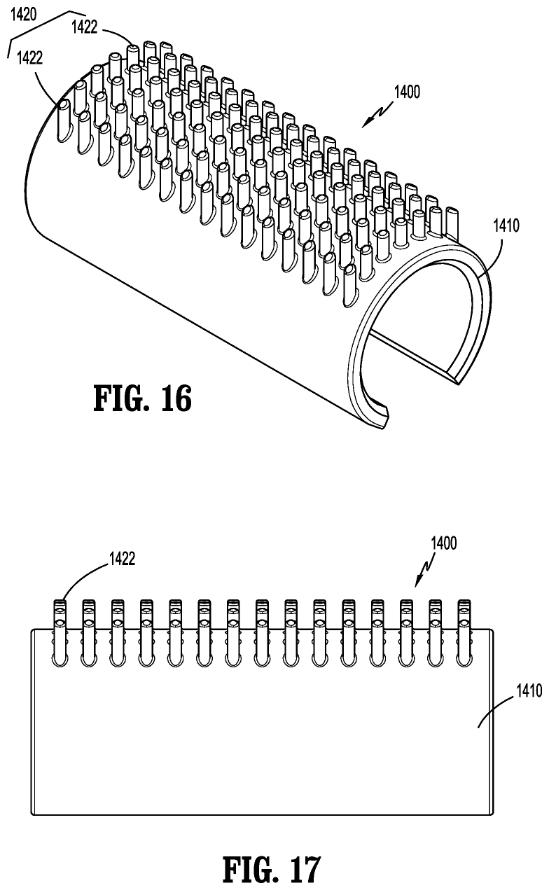

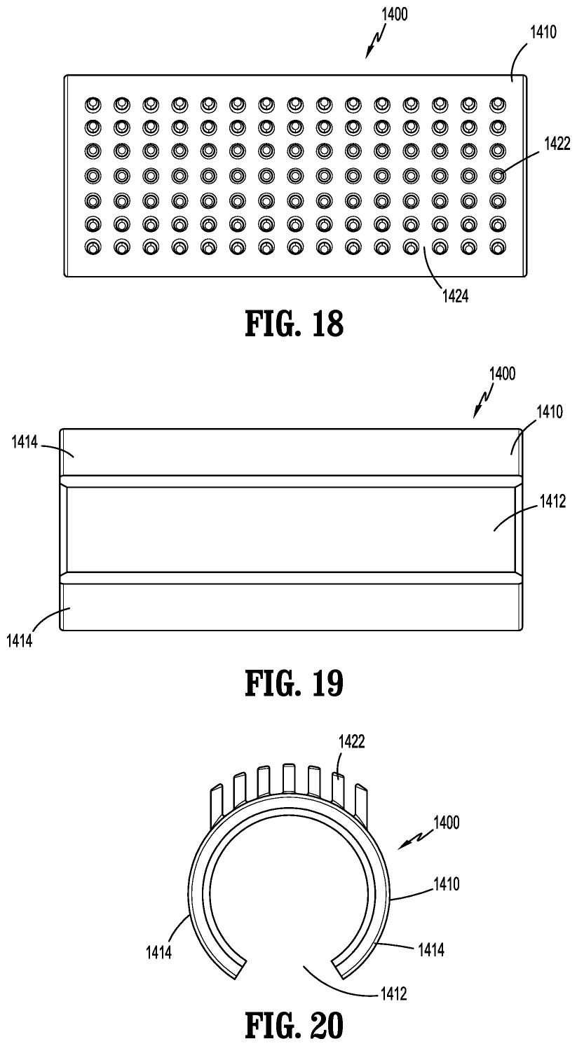

FIGS. 16-20 illustrate the cleaning device 1400 including a clamp body 1410 having an external part 1420 extending from the clamp body 1400. The external part 1420 includes a plurality of protuberances 1422 that extends in the same general direction and partially arranged around a circumference of the clamp body 1410. The plurality of protuberances 1422 may have same dimensions such that the plurality of protuberances 1422 generally conforms to a curvature of the clamp body 1410. Further, adjacent protuberances 1422 may define a gap 1424. In this manner, the plurality of protuberances 1422 may engage the lens 18 (FIG. 4) of the endoscope 10 and remove debris from the lens 18. The debris may be collected in the gap 1424. The clamp body 1410 has an arcuate configuration having, e.g., a C-shaped profile. The clamp body 1410 is formed of a resilient material to enable the clamp body 1410 to be detachably snapped on to the elongate shaft 210 (FIG. 14) of the surgical clip applier 200. In particular, the clamp body 1410 defines a slot 1412 between flexible wings 1414 to enable passage of the elongate shaft 210 through the slot 1412. Once the elongate shaft 210 is received through the slot 1412, the flexible wings 1414 engage the elongate shaft 210 to detachably secure the cleaning device 1410 to the elongate shaft 210. While the plurality of protuberances 1422 is shown, it is contemplated that any of the external parts shown hereinabove, may be used with the clamp body 1410.

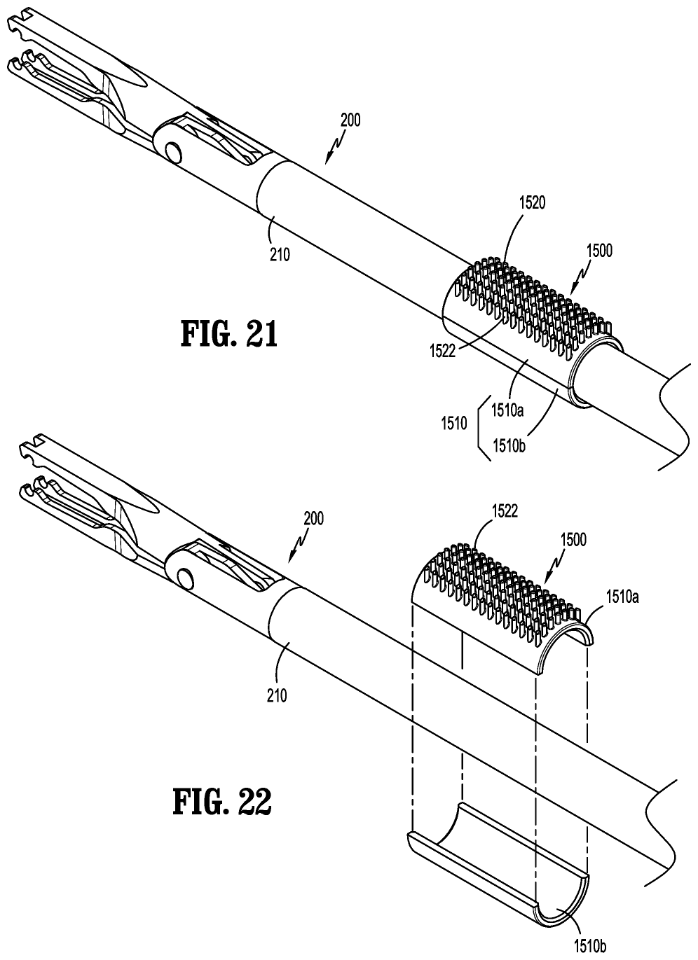

FIGS. 21 and 22 illustrate a cleaning device 1500 in accordance with another aspect of the disclosure. The cleaning device 1500 includes a sleeve 1510 having first and second halves 1510a, 1510b that are coupled to be mounted on the elongate shaft 210 of the surgical clip applier 200. The cleaning device 1500 includes an external part 1520 including a plurality of protuberances 1522 extending from the first half 1510a of the sleeve 1510 in a same general direction. The external part 1520 may be any one of the external parts described hereinabove. The first and second halves 1510a, 1510b of the sleeve 1510 may be detachably coupled to each other by, e.g., magnetic coupling. For example, magnetic coupling would allow different cleaning devices to be provided in a kit so that the clinician may select one for use. Alternatively, the first and second halves 1510a, 1510b may be fixedly coupled to the elongate shaft 210 by, e.g., ultrasonic welding or glue.

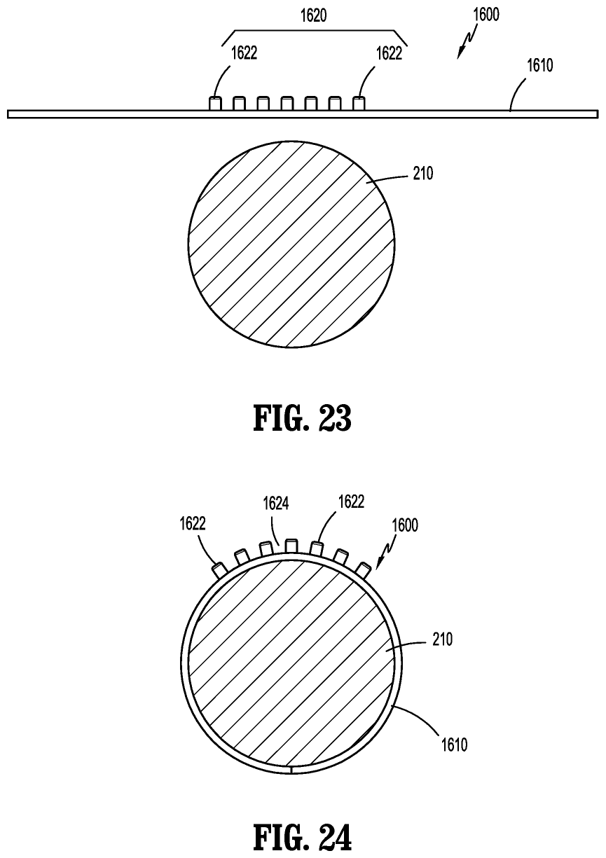

FIGS. 23 and 24 illustrate a cleaning device 1600 in accordance with yet another aspect of the disclosure. The cleaning device 1600 includes a supporting base 1610 and an external part 1620 extending from the supporting base 1610. The supporting base 1610 is formed of, e.g., a malleable material, to be transitionable between a flat configuration and a bent configuration conforming to an external surface of the elongate shaft 210 to secure the cleaning device 1600 to the elongate shaft 210 of the surgical clip applier 200. The external part 1620 includes a plurality of protuberances 1622 configured to wipe the lens 18 (FIG. 4) of the endoscope 10 to remove debris from the lens 18. Adjacent protuberances 1622 may define a gap 1624 to collect debris from the lens 18. However, it is contemplated that the external part 1620 may be any one of the external parts identified above. Use of the cleaning devices 300, 400, 500, 600, 700, 800, 900, 1000, 1100, 1200, 1300, 1400, 1500 are substantially similar to use of the cleaning device 100 as described hereinabove, and thus, will not be described herein. It is also envisioned that the cleaning devices 100, 300, 400, 500, 600, 700, 800, 900, 1000, 1100, 1200, 1300, 1400, 1500 may be adapted for use with a robotic surgical system.

While the disclosure has been shown in the drawings, it is not intended that the disclosure be limited thereto, as it is intended that the disclosure be as broad in scope as the art will allow and that the specification be read likewise. Therefore, the above description should not be construed as limiting, but merely as exemplifications. Those skilled in the art will envision other modifications within the scope and spirit of the claims appended hereto.

What is claimed is:

1. A surgical kit comprising:
a surgical instrument including an elongate shaft and an end effector mounted on a distal end portion of the elongate shaft;
an endoscope having a lens at a distal end portion of the endoscope; and
a cleaning device including:
a sleeve defining a lumen dimensioned to receive the end effector and the elongate shaft therethrough, the sleeve being flexible to be mountable about the elongate shaft of the surgical instrument; and
an external part disposed on the sleeve to remove debris from the lens of the endoscope when the lens of the endoscope is wiped against the external part, the external part defining a gap to collect debris on the lens of the endoscope.

2. The surgical kit according to claim 1, wherein the external part of the cleaning device includes a plurality of ribs on the sleeve, the plurality of ribs defining an acute angle with respect to a longitudinal axis defined by the sleeve.

3. The surgical kit according to claim 1, wherein the external part is formed of an elastomer.

4. The surgical kit according to claim 1, wherein the sleeve is formed of an elastomer.

5. The surgical kit according to claim 1, wherein the cleaning device is integrally formed as a single construct.

6. The surgical kit according to claim 1, wherein the cleaning device is monolithically formed.

7. The surgical kit according to claim 2, wherein each rib of the plurality of ribs is separate from other ribs.

8. The surgical kit according to claim 1, wherein the external part is a rib having a continuous helical configuration.

9. The surgical kit according to claim 1, wherein the external part extends around a portion of a circumference of the sleeve.

10. The surgical kit according to claim 1, wherein the external part is arranged along a length of the sleeve.

11. The surgical kit according to claim 2, wherein each ribs of the plurality of ribs is tapered or beveled and includes a wiping portion configured to engage a surface of the lens of the endoscope to remove debris from the lens.

12. The surgical kit according to claim 1, wherein the external part is a plurality of protuberances extending in a same general direction.

13. The surgical kit according to claim 12, wherein the plurality of protuberances conforms to the contour of the sleeve.

14. The surgical kit according to claim 13, wherein the plurality of protuberances defines a plane to engage a surface of the lens of the endoscope.

15. The surgical kit according to claim 12, wherein adjacent protuberances define the gap therebetween to collect debris from the lens of the endoscope.

16. The surgical kit according to claim 12, wherein the sleeve and the plurality of the protuberances are formed of different materials.

17. A surgical kit comprising:
a surgical instrument including an elongate shaft and an end effector mounted on a distal end portion of the elongate shaft;
an endoscope having a lens at a distal end portion of the endoscope; and
a cleaning device including:
a clamp body mountable on the elongate shaft of the surgical instrument, the clamp body being flexible and defining a slot to receive the elongate shaft therethrough to secure the clamp body on the elongate shaft; and
an external part disposed on the clamp body to remove debris from the lens of the endoscope when the lens of the endoscope is wiped against the external part.

18. The surgical kit according to claim 17, wherein the external part includes a plurality of protuberances partially arranged around a circumference of the clamp body.

19. A surgical kit comprising:
a surgical instrument including an elongate shaft and an end effector mounted on a distal end portion of the elongate shaft; and
a cleaning device including:
a base transitionable between a first configuration, in which, the base defines a planar surface, and a second configuration, in which, the base conforms to an exterior surface of the elongate shaft of the surgical instrument to mount the base to the surgical instrument; and
a plurality of protuberances extending from the base to remove debris from a lens of an endoscope when the lens of the endoscope is wiped against the plurality of protuberances.

20. The surgical kit according to claim 19, wherein the plurality of protuberances is formed of silicone or rubber.
